(19) United States
(12) Patent Application Publication
(10) Pub. No.: US 2012/0109071 A1
(43) Pub. Date: May 3, 2012

(54) MEDICAL DELIVERY DEVICE WITH TIME LAPSE INDICATOR

(75) Inventors: Andre Larsen, Dragor (DK); Salim Bouaidat, Copenhagen K (DK)

(73) Assignee: Novo Nordisk A/S, Bagsvaerd (DK)

(21) Appl. No.: 13/318,253

(22) PCT Filed: Apr. 29, 2010
(86) PCT No.: PCT/EP2010/055852

§ 371 Date: Jan. 16, 2012

Related U.S. Application Data
(60) Provisional application No. 61/176,318, filed on May 7, 2009.
(60) Provisional application No. 61/255,673, filed on Oct. 28, 2009.

(30) Foreign Application Priority Data
May 2, 2009 (EP) 09159288.1
Oct. 14, 2009 (EP) 09172958.2

(51) Int. Cl. A61M 5/31; B05D 5/00

(52) U.S. Cl. 604/189; 427/2.1

(57) ABSTRACT

The present invention relates to medical delivery devices for administering a dose of a drug into the body of a subject user and incorporating a time lapse indicator which is configured for indicating after administration that a dose of the drug actually has been administered and for maintaining this indication until lapse of a pre-defined time interval. The time lapse indicator disclosed is based on a pressure sensitive substance which exhibits a change in a visual property in response to an action associated with administering a dose and wherein the pressure sensitive substance exhibits a further change in a visual property after lapse of a pre-defined time interval subsequent to said action.

### FIELD OF THE INVENTION

The present invention relates to medical administering devices for administering a drug into the body of a subject user wherein an indicating device is configured to indicate, subsequent to an administration, that a dose of the drug has been administered and for maintaining this indication until lapse of a pre-defined time interval.

### BACKGROUND OF THE INVENTION

Some medication, such as insulin, is typically self-administered using a medical delivery device such as an injection pen. The typical diabetes patient will require injections of insulin several times during the course of a week or a day. However, typical injection devices do not address the problem of a user not remembering when the last injection was administered.

Even shortly after administering a dose of insulin, the user now and then will be in doubt as to whether he actually carried out an injection or not. This could be after minutes or even hours after the intended time for performing an administration. Thus, there exists the potential hazard that the patient chooses not to take his medication or that he takes it twice.

Some prior art devices, such as the electronic syringe disclosed in WO 97/30742, are provided with an electronic monitoring system adapted to automatically start an electronic timer when a selected dose is injected and to show the progress in time on an electronic display. Such an injection device generally provides a satisfactorily solution to the problem addressed above. However, for simpler devices such as disposable injection devices, i.e. the so-called pre-filled devices, the solution with integrated electronics will in most cases not be economically viable. In addition, such a solution may not be environmentally acceptable due to the potential increase in the disposal of electronic components such as batteries etc.

WO 99/43283 include disclosure of a timer device which is intended to be used with pre-filled injection pens, where the timer device is configured for releasable attachment to the pre-filled pen so that the timer device can be removed from a pen once it is ready for disposal and be attached to a new pen. The timer device is configured to detect when an injection is performed and to communicate this via indicator lights that remains turned on for a certain time period from the administration of the dose.

WO 02/064196 includes disclosure of a related timer device, however being more sophisticated in respect of the features it provides.

Although disclosing environmentally acceptable solutions, both the devices of WO 99/43283 and WO 02/64196 offer somewhat complex solutions which require additional operational measures from the user before they can be used. In addition, the device of WO 99/43283 offers a rather bulky design in that it is attached to the rear housing of an injection pen so as to surround it and further requires a second part which couples to the injection button of the device to monitor the movements of the injection button.

In a further reference, WO 2007/134067, a vascular access device is disclosed, the device being provided with a status indicator which includes a piezochromic material to indicate proper disinfective swabbing of a septum, encouraging an operator to disinfect the entire surface of the corresponding status indicator.

### BRIEF DESCRIPTION OF THE INVENTION

Having regard to the above-identified prior art, it is an object of the present invention to provide a timer device solution for a medical administering device or a medical delivery device which provides an elapsed time indicator of a less complex construction and which do not require an electric circuitry. A further object is to provide a simple and cost-effective solution so that the time lapse indicator will be adaptable for inclusion as an integral part of a disposable device such as a pre-filled medical delivery device and which enables an easier operation of the device.

In a first aspect the present invention relates to a handheld medical administering device, such as a drug administering device, for accommodating a drug and for administering one or more doses of a drug to a subject user, the medical administering device comprising:
means for the administering of a dose of the drug from the device, and
a time lapse indicator adapted to be in a first state prior to an action associated with the administering of the dose, to change to a second state during or subsequent to said action and to change to a third state after lapse of a pre-defined time interval subsequent to said action,
wherein the time lapse indicator comprises a pressure sensitive substance exhibiting a change in a visual property when exerted to pressure and/or force and wherein the medical delivery device is so configured that pressure is exerted upon the sensitive substance responsive to a user performing the said action.

The first, second and the third state of the time lapse indicator may respectively exhibit first, second and third visual states, where the state changes from the first state to the second state and from the second state to the third state will be readily observable by the human eye.

By incorporating a time lapse indicator of the above kind into an administering device, a user may easily check whether the device has been used for administering within the course of the preceding pre-defined time interval. Hence, if a user do not feel confident about whether he or she is in proper compliance with the personal treatment plan, the time indicator may be used to improve confidence and ultimately be used for a proper corrective action if needed. The above solution forms a simple and reliable indicator enabling an easy incorporation into a medical administering device. Furthermore, when being built into the medical delivery device as an integral part, a particularly user-friendly solution is provided which do not require any user involvement in the operation of the timer indicating function.

In particular embodiments, the pressure sensitive substance is adapted to alter one or more properties selected from the group consisting of colour, absorptivity, reflectivity, transmissivity, diffusivity, and polarization when being exerted to a change in pressure and/or force. Within the context of the present invention, additional or alternative optical property changes can be incorporated in the time lapse indicator as well without departing from the scope of the invention.

The pressure sensitive substance may be reversible so that the indicator returns to the initial visual state upon lapse of the pre-defined time interval. Also, the pressure sensitive substance may be reactivatable such that the pressure sensitive substance may be brought into the second state more than once when exerted to repeated activation. Hence, in some embodiments, the pressure sensitive substance may be brought from the first state to the second state more than once or from the third state to the second state more than once. The pressure sensitive substance may be so selected that the pre-defined time interval is initialised by subjecting the pressure sensitive substance to a change in pressure or force, such as upon exertion of pressure/force or upon release of said pressure/force. In some embodiments, the pressure sensitive substance is selected so that the time lapse indicator, when being brought into the second state, by renewed action such as by a further actuation of an actuator, prolongs the time interval by a duration equal to the pre-defined time interval.

The pressure sensitive substance may be so chosen as to substantially exhibit a uniform visual appearance throughout the course of the pre-defined time interval so that a particular marked change in the visual appearance upon lapse of the pre-defined time interval is obtained.

The time lapse indicator of the present invention may be designed so that the pre-defined time interval is longer than 5 minutes, such as longer than 15 minutes, such as longer than 30 minutes, such as longer than 1 hour, such as longer than 2 hours, such as longer than 5 hours, such as longer than 10 hours, such as longer than 20 hours, such as longer than 36 hours.

The pressure sensitive substance may comprise a piezochromic material. Said piezochromic material may be reversible so that the indicator returns to the initial visual state upon lapse of the pre-defined time interval.

A non exhaustive list of usable materials include bianthrones, spiropyrans, salicylidene anilines, fluoran compounds, metal cluster compounds and copper complexes. A reversible indicator may comprise a piezochromic system comprising an electron donating compound and an electron accepting compound. The electron donating compound is an ionochromic substance which is a pH-sensitive dye and the electron accepting compound acts as the colour developer. The developer has acidity strong enough to change the colour of the ionochromic substance by protonation, yet weak enough to allow the system to remain reversible. Suitable ionochromic compounds are electron donating compounds such as pH-sensitive dyes, preferably leuco-dyes, or other colour formers. Most commonly used often belong to the spirolactone class. The protonation of a colourless or substantially colourless lactone by a weak-acid developer causes the lactone ring to open and results in a formation of a coloured compound.

The piezochromic materials may further be selected as one or more of the materials disclosed in US patent application No. 2007/0259286, in particular the materials mentioned in Table 3 of this reference, this reference being incorporated herein in its entirity.

In other embodiments, the pressure sensitive substance is selected as one or more of the materials disclosed in US 2007/0172951, U.S. Pat. No. 4,687,862, U.S. 6,261,469, U.S. 5,320,784 and U.S. 5,501,945. Other embodiments may encompass any feature as listed by the appended claims.

In particular embodiments, the administering device includes an actuator and an outlet, wherein the actuator is used for delivering a dose of the drug from an outlet of the administering device.

In particular embodiments, the medical administering device is a device for self-administering one or more doses of a medicament-containing drug.

In exemplary embodiments, the action that causes the time lapse indicator to change from the first to the second state is selected from actions which during normal or intended use are considered necessary for performing an administration, i.e. bringing the drug into the body of the user or dispensing the drug in a form to be readily brought into the body. Depending on the design of the specific administering device, the administering action may be selected from the group consisting of a) actuating an actuator, b) operating a dose size selector, c) moving a cap from an outlet covering position to an outlet uncovering position or vice versa, d) changing the relative position between a needle shield and an injection needle, i.e. exposing the needle or covering it after exposure e) arming the device for subsequent actuation, f) releasing a lock enabling operation of the device, and g) operating a mechanism for the dispensing of a pill or tablet from a medicament dispenser, such as in a pill dispenser, and h) operating a mechanism for gaining access to a dose in a medicament dispenser.

For example, when the action associated with the administering action is defined as the removal or reattachment of a cap with respect to a drug outlet, the cap may form, when the device is a medical injecting device, a cover for covering an injection needle, or alternatively, when the device is a medical inhaling device, it may form a cover for covering a mouthpiece of the device.

In embodiments where the drug administering device includes an actuator for injecting a dose of a drug, said action may be defined as the actuation of the actuator. In such a device, the pressure sensitive substance is configured so that actuation of the actuator causes the pressure sensitive substance to change from the first state to the second state. In some embodiments, the actuator is adapted to move in a distal direction when actuated. The pressure sensitive substance may be arranged on a proximally facing part of the actuator.

In embodiments where the drug administering device includes a dose size selector which is operable to set the size of a dose, said action may be defined as the operation of the dose size selector. The pressure sensitive substance may be so configured that operation of the dose size selector causes the pressure sensitive substance to change from the first state to the second state.

In other embodiments where the drug administering device includes a cap which can be moved from a protecting state where the outlet is protected during storage of the device to a non-protected state where the outlet is exposed for administering a dose of the drug, the said action may be defined as the moval of the cap from the protecting state to the non-protecting state and/or from the non-protecting state to the protecting state.

Still other embodiments of drug administering devices include an outlet comprising a needle mount for connecting to an injection needle, wherein the drug administering device further comprises a needle shield for shielding a needle, when mounted on the drug administering device. The needle shield may be configured to move relative to the needle when the injection needle is inserted into and/or removed from the skin of the subject user and said action is defined as the said insertion and/or removal of the injection needle into/from the skin of the subject user.

In one form, the medical administering device forms a unit, wherein the drug is accommodated within a housing of the device so that the device forms a unit which can be grasped by the hand of the user. Such unit may be shaped and sized so as to be held in a pocket. In particular embodiments, the drug is accommodated in a cartridge and the cartridge is received or held within the device so that the complete device forms a handheld unit which can be grasped by the hand of the user.

When the device includes a cartridge, or is adapted to hold a cartridge which the user couples to the housing of the device before administration, the cartridge may be of the type including a movable plunger which is slideably adjustable inside a form-stable portion of the cartridge housing, or alternatively, the cartridge may be of the collapsible type which includes at least one wall section which collapses as the drug is expelled or sucked from the cartridge.

The medical administering device may be of the manual type, where a user, during injection, supplies the force necessary for expelling medicine from the reservoir. Alternatively, the medical delivery device may be of the kind commonly known as a wind-up pen or AutoPen®, e.g. an injection device where the user, during an initial procedure, delivers the mechanical energy required for the expelling operation, the mechanical energy being stored as potential energy for example in a spring member.

In some embodiments, the administering device may be adapted to administer a single fixed sized dose or a single user-selectable dose of a drug so that the device forms a single-administration device to be disposed off after administration of a single dose. In such a device the pressure sensitive substance may be adapted to transform into a third state after lapse of the pre-defined time interval, the third state being different from the first state. In other embodiments, the third state may visually correspond to the first state.

In other embodiments, the administering device may be adapted to administer a series of distinct doses of a drug, wherein each administered dose may be pre-fixed (i.e. not user adjustable) or wherein the size of the administered doses is user-selectable. In such devices, the third state may visually correspond to the first state.

In one embodiment, the administering device is a syringe having a slideable piston which performs as an actuator and where the time lapse indicator is arranged on an actuatable part of the piston.

In other embodiments, the administering device is a medical doser, an injection pen, a jet injection apparatus or an inhaling device. In still other embodiments, the administering device is a pill or tablet dispenser, where the time lapse indicator is associated with a part which moves for gaining access to one or more pills or tablets, so that a person gaining access to one or more pill(s) or tablet(s) activates the time lapse indicator automatically when gaining access or closing off a pill or tablet compartment.

The time lapse indicator including the pressure sensitive substance may be disposed on the administering device as a label which is adhered to a component of the device. Alternatively, the pressure sensitive substance is disposed directly onto a component of the device, such as by printing or coating onto the particular component. Still alternatively, the pressure sensitive substance may be formed integral with a component of the device, e.g. as compounded into a plastic material and molded to form the particular component which is to include the time lapse indicator. In some embodiments, the component having the pressure sensitive substance as an integral component may be made of an elastomeric material.

In some embodiments, the time lapse indicating portion is positioned next to a permanent indicator for comparison of the visual appearance of the time lapse indicator with one or more reference fields.

In embodiments where the time lapse indicator is adapted to removably attach to a medical administering device, such removable attachment may be provided as a mechanical coupling to the actuator of the delivery device. Suitable attachments may be provided as a snap-coupling, a threaded coupling or a bayonet coupling. Other removable attachments may be provided by an adhesive or by using fasteners such as Velcro™.

In other embodiments, the time lapse indicator comprises a plurality of different time lapse indicators each having different time delay properties so that the time lapse indicator may be used to signal a plurality of different time expirations after an action which activates the initial change from the first visual state to the second visual state. Such indicators may be arranged to form multi-segmented displays defining multiple separate areas which visually changes state at different times after activation. Also, a plurality of time lapse indicators may be intermeshed so as to provide an appearance as a contiguous area which are able to change between numerous different colors, such as three, four or more colors.

In a second aspect, the present invention relates to an injection needle assembly for cooperation with a medical delivery device having a time lapse indicator of the same kind as described in connection with the first aspect for signalling whether or not the injection needle has been used, i.e. inserted into the skin of the subject user, within the course of the preceding pre-defined time interval.

In particular embodiments, the injection needle assembly comprises a needle cannula having a distal end for penetrating the skin of a subject user and a needle shield, wherein the needle assembly is positionable in a shielded state where the distal end of the needle cannula is shielded by the needle shield and in an exposed state where the distal end of the needle cannula is exposed for insertion into the skin of the subject user. A visual indicator is adapted to change from a first state to a second state responsive to the needle assembly changing from said shielded state to the exposed state or vice versa, wherein the visual indicator is configured for indicating time elapsed since said change from the first state to the second state. The visual indicator incorporates a pressure sensitive substance exhibiting a change in a visual property when exerted to pressure or force. The visual indicator changes said visual property subject to the needle assembly changing from the shielded state to the exposed state or vice versa. The pressure sensitive substance exhibits a further change in a visual property after lapse of a pre-defined time interval subsequent to said change from the first state to the second state.

In a third aspect, the present invention relates to the use of a pressure sensitive substance in a time lapse indicator associated with an administering device as described herein.

In a fourth aspect, the present invention relates to a method of manufacturing a medical having a time lapse indicator as described herein.

In a fifth aspect, the present invention relates to a blister package having one or more blister cavities for accommodating or accommodating an article or a portion of bulk or fluid material in each of said one or more blister cavities, each of said one or more blister cavities comprising a first material portion defining at least one cavity, and a lid portion closing off said at least one cavity. The blister package comprises a time lapse indicator being adapted to change responsive to being exerted to pressure and/or force from a first state to a second state and to change to a third state or return to the first state after lapse of a pre-defined time interval subsequent to said application of pressure and/or force. Hence, subsequent to a user rupturing a blister cavity, the user may retrospectively check whether or not an administration has been performed within the lapsed period of time corresponding to the pre-defined time interval.

The time delay indicator of the fifth aspect may comprise a visual indicator wherein the visual indicator changes a visual property when changing from said second state to said third state or changing from said second state to said first state. The time delay indicator may comprise a pressure sensitive substance exhibiting a change in a visual property upon lapse of said pre-defined time interval. Any of the features mentioned above in connection with the time lapse indicator of the first aspect may, as long as compatible with the blister package of the fifth aspect, be incorporated in a blister package.

In an embodiment said time lapse indicator is associated with at least one of said blister cavities so that the time lapse indicator is activated upon a user gaining access to the article or portion of bulk or fluid material in said cavity. Such indicator may be pressed or squeezed by the user upon dispensing from each of said blister cavities, either by the dispensing activity or by an action separate from said dispensing activity.

In further embodiments, the blister package includes a plurality of distinct time lapse indicators so that a respective time lapse indicator is associated with each of said one or more blister cavity/cavities

In a further embodiment, the blister package comprises a plurality of blister cavities but a single time lapse indicator which is adapted to be reactivated such that it may be activated more than once to change from said first state to said second state upon administration of the contents of each respective blister cavity.

The lid of said blister package may be formed as a planar sheet, or alternatively define a second cavity positioned to mate with the corresponding cavity/cavities formed in the first material portion, the cavity/cavities of the lid either facing or facing away from the cavity/cavities of the first material portion. In some embodiments, the first material portion and the lid may be formed as contiguous sheets of materials that are bonded together or, alternatively, the first material portion and the lid may be formed by the same sheet of material which is folded along an edge and then bonded. In embodiments where the blister package forms a plurality of blister cavities, the first sheet portion or, alternatively, the lid may be formed as individual portions which are mutually spaced relatively to each other.

The time lapse indicator(s) of the blister package may be associated with the first material portion and/or the lid, e.g. formed on or in the sheet materials of the first material portion or the lid. In this way the particular time lapse indicator in question is activated when the associated blister cavity is accessed, e.g. when one of the layers associated with a blister cavity is ruptured.

By incorporating a time lapse indicator of the above kind into a blister package according to the fifth aspect, a user may easily check whether the blister package has been used for dispensing within the preceding pre-defined time interval.

For example, when the blister package holds one or more portions of a medicament, the time indicator may be used by a patient to ensure whether or not a dispensing has been performed within an elapsed time interval corresponding to the preceding pre-defined time interval. Hence, if a user do not feel confident about whether he or she is in proper compliance with the personal treatment plan, the time indicator may be used to improve confidence and ultimately be used for a proper corrective action if needed. The above solution forms a simple and reliable indicator enabling an easy incorporation into a medical blister package. Furthermore, when being built into the blister package as an integral part, a particularly user-friendly solution is provided which do not require any user involvement in the operation of the timer indicating function.

In the context of the present invention, the term “medical administering device” shall be understood as any device capable of passively or actively bringing a medicament-containing drug into the body of a user by means of an appropriate dispensing mechanism, either transdermally, orally or nasally. A non-exhaustive list of medical administering devices within the context of the present invention comprises medical delivery devices as well as medicament dispensers such as pill or tablet dispensers or blister packages.

In the context of the present invention, the term “medical delivery device” shall be understood as any device capable of actively bringing a medicament-containing drug into the body of a user by means of an appropriate delivery mechanism, such as either transdermally, pulmonary or nasally. A non-exhaustive list of medical delivery devices within the context of the present invention comprises pre-filled or durable injectors such as pen-shaped injectors, dosers, inhalators or infusion pumps. The drug may be flowable, e.g. to be administered with an injection needle or by jet-injection, or solid such as drugs forming medicine pegs for insertion through the derma. Representative medicaments includes pharmaceuticals such as peptides, proteins (e.g. insulin, insulin analogues and C-peptide), and hormones, biologically derived or active agents, hormonal and gene based agents, nutritional formulas and other substances in both solid (dispensed), gaseous or liquid form.

In the context of the present invention, the term “pressure sensitive substance” shall be understood as any substance or composition which exhibits a change in a visual property when exerted to pressure and/or force. The term “pressure sensitive substance” encompasses both a single substance or material which exhibits the above change in a visual property when exerted to pressure and/or force as well as a combination of substances which interacts under the influence of pressure and/or force to exhibit a change in a visual property.

The term “subject user” encompasses human beings as well as animals.

The term “handheld medial delivery device” encompasses both devices adapted to be held in a hand when operating, as well as patch devices being adapted to be fixed to a subject user during operation.

### DETAILED DESCRIPTION OF THE INVENTION

The invention will now be described in further detail with reference to the drawings in which:

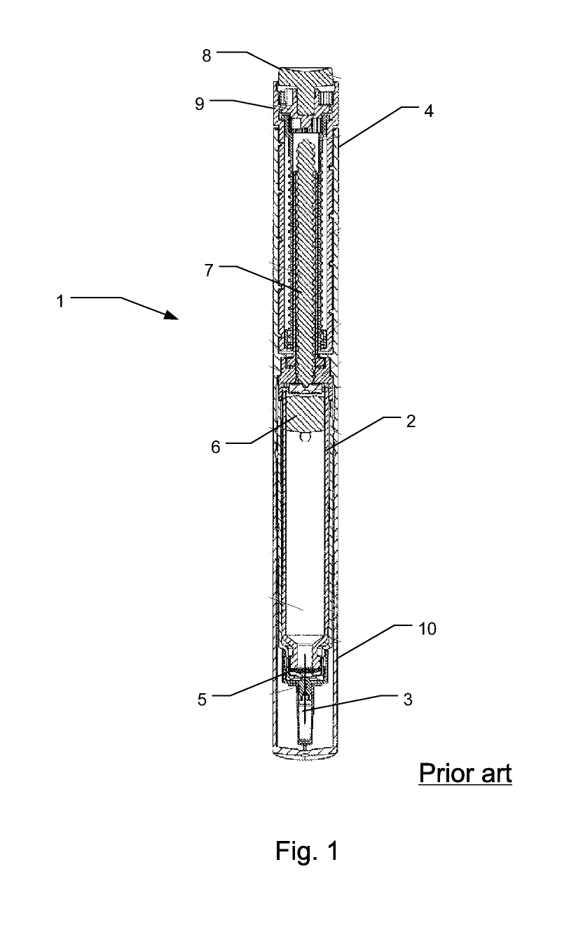

FIG. 1 shows a prior art medical delivery device,

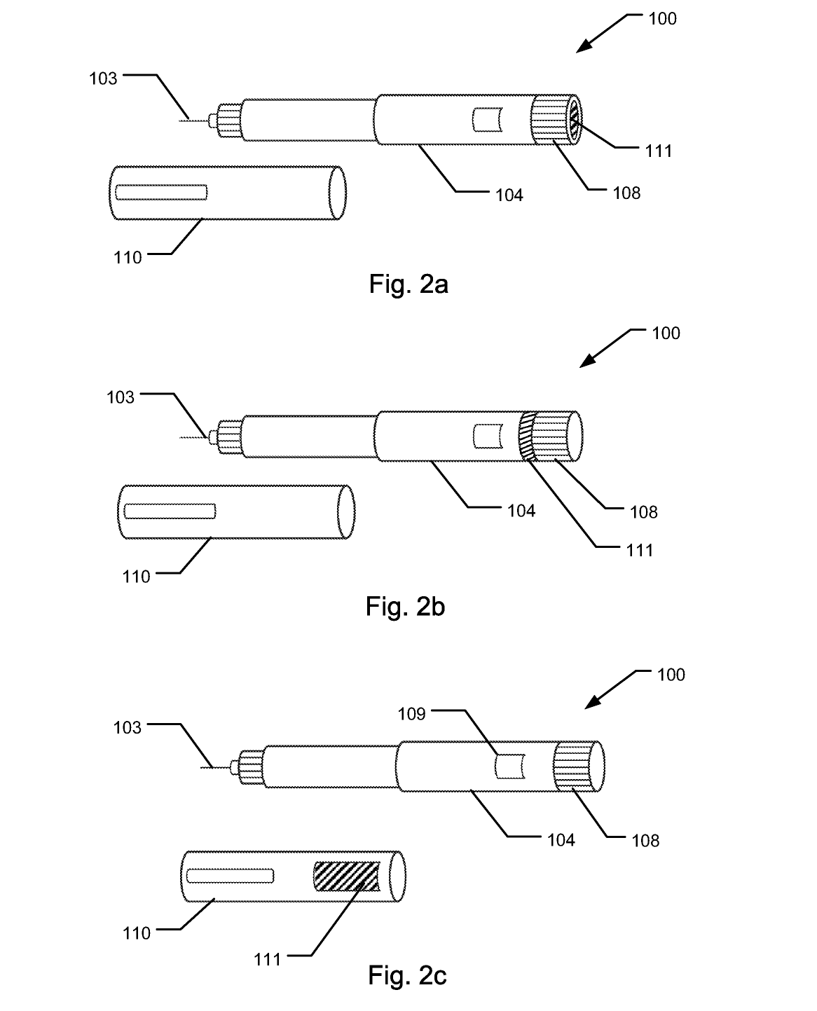

FIGS. 2a, 2b and 2c are schematic representations respectively showing medical delivery devices corresponding to first, second and third embodiments according to the present invention,

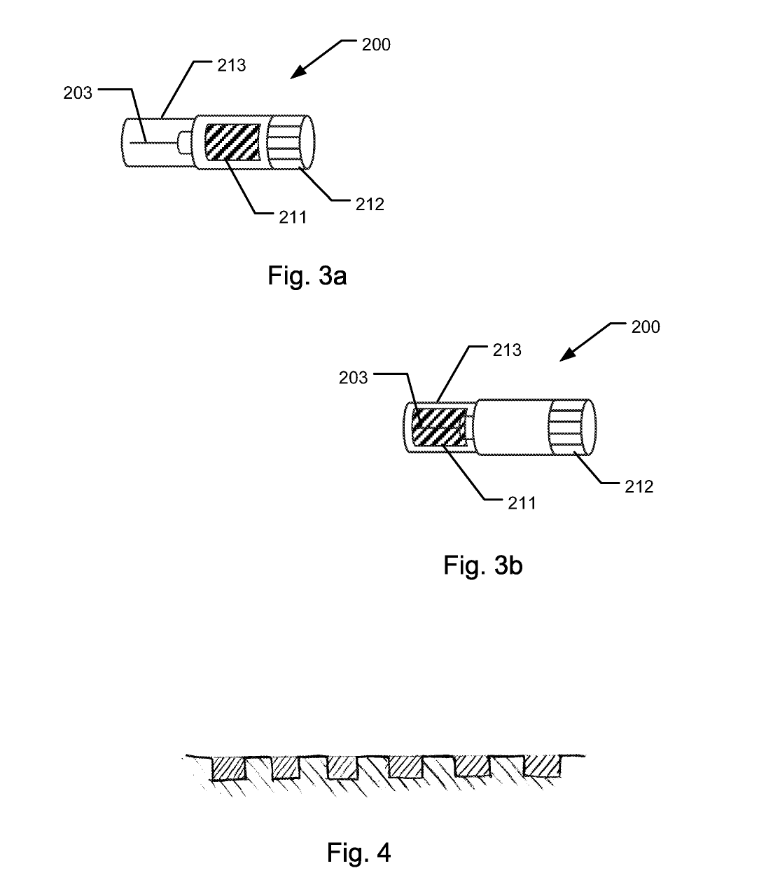

FIGS. 3a and 3b are schematic representations respectively showing needle assemblies corresponding to fourth and a fifth embodiments according to the present invention,

FIG. 4 show a schematic representation of a time lapse indicator having several state changes,

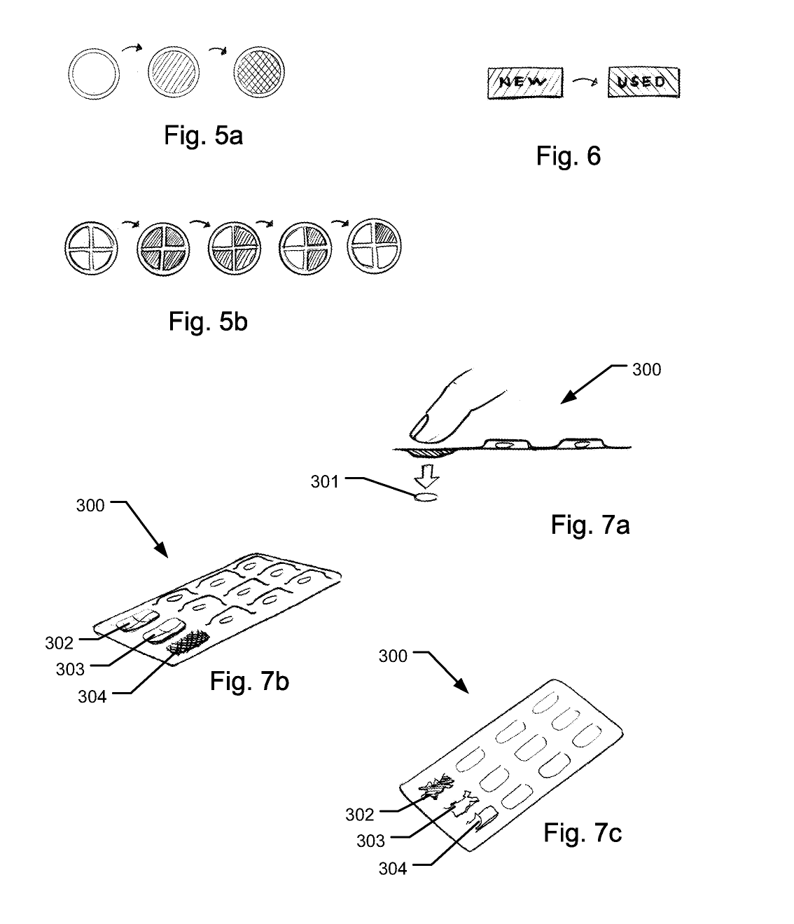

FIGS. 5a and 5b shows different time lapse indicators having several state changes,

FIG. 6 show a time lapse indicator having two distinct messages, and

FIGS. 7a, 7b and 7c show a blister package including time lapse indicators.

FIG. 1 discloses a prior art medical delivery system forming an injection pen 1 comprising a medicament filled cartridge 2 which is accommodated in a distal part of the pen. A main housing part 4 of the device holds a mechanism for setting and injecting specific doses of a medicament from the cartridge 2. The cartridge 2 comprises a passage in a distal neck part which is sealed by a piercable sealing member 5. The cartridge further comprises a slideably mounted piston 6 which is adapted to slide towards the distal part of the container 2 when a force is exerted on the piston 6 in the distal direction. During administration, medication is delivered through an outlet, which in the depicted form is shown as an injection needle assembly including a needle cannula 3, the injection needle assembly being releasably secured to the distal part of the pen. In the depicted form, a dose size selector 9 in the form of a dose dial knob is turned for setting a proper dose size. The injection pen further comprises an actuator 8 in the form of an injection button which can be pressed forward in the distal direction to inject the set dosage. A distally directed force on the button 8 exerted by the hand of the user is transferred by the dosing mechanism 7 of the pen to the piston 6 of cartridge 2. The injection device further comprises a detachable cap 10 which is used for protecting the part of the device which accommodates the cartridge 2 and for protecting a needle assembly which is secured to the remainder of the pen.

FIG. 2a shows schematic representation of a first embodiment of a medical delivery device according to the present invention. In the depicted form, an injection pen 100 for injecting one or more apportioned doses of a drug contained within the injection pen is arranged with a main housing 104 incorporating a dosing mechanism and accommodating a drug to be administered. An injection needle assembly including a needle cannula 103 is connected to a distal portion of main housing 104. A cap 110 is shown in its detached or non-protecting state where the needle assembly is exposed. A proximal part of the device incorporates an actuator 108 which can be actuated for delivering a dose of a drug from the device. Actuator 108 is manually pushable from a proximal position to a distal position in order to actuate actuator 108. A proximal portion of actuator 108, such as the proximal face of actuator 108, includes a time lapse indicator 111 which comprises a pressure sensitive substance which changes visible appearance when subject to pressure. In the depicted embodiment, the pressure sensitive substance is a piezochromic substance which exhibits a change in colour when exerted to pressure or strain. Hence, when a user administers a dose of the drug from the injection pen 100, the applied pressure of a finger pushing the proximal face of actuator 108 causes the piezochromic substance to change colour, indicating that an administration has been carried out. The Piezochromic substance is chosen so as to provide a reversible indicator which upon release of the pressure returns to the initial colour after a specific delay. By appropriately selecting the type of piezochromic substance, the return to the initial colour occurs after lapse of a given time interval following release of pressure. Example time intervals may be chosen in the order of 30-60 minutes or even a plurality of hours. By checking the colour of the piezochromic material, a user of the injection pen may assure him or herself whether a planned administration has been carried out or not.

In the first embodiment shown in FIG. 2a, the piezochromic material has been applied directly on a proximal face of actuator 108. In alternative embodiments, the actuator 108 comprises a proximal transparent section which may be designed to cover the piezochromic substance, so that the substance can be viewed through the transparent section. The transparent section may be arranged to be relatively moveable with respect to the remaining part of actuator 108 to move along the axis of the actuating direction of the actuator so that the piezochromic material becomes squeezed or otherwise manipulated underneath the transparent section as the transparent section is pressed towards the remaining part of the actuator 108. In this way, by appropriately designing the actuator, the applied pressure may be exerted uniformly across the area of the piezochromic substance for increased consistency in the change of colour. Also, in order to ensure that a large pressure is exerted upon the piezochromic substance, appropriate design of the actuator 108 may ensure that only the area of the piezochromic substance or primarily that specific area actually transfers the force which is applied on the actuator in the distal direction. Hence by selecting a relatively small area of piezochromic substance, compared to the proximal area of the actuator, a large contact pressure may be ensured.

FIG. 2b shows a second embodiment which is a slightly modified version of the first embodiment shown in FIG. 2a. In this embodiment, the time lapse indicator 111 including the piezochromic material is arranged on a proximal face of the main housing 104. Actuator 108 is adapted to engage the piezochromic substance when the actuator 108, during dose administration, is fully pushed in. Due to the pressure which is exerted by actuator 108, the piezochromic substance changes colour for a pre-defined time-interval subsequent to the action of administering the dose.

In a further not shown embodiment, which largely corresponds to the embodiment shown in FIG. 2b, the actuator 108 may be dimensioned so as to fully or partly encircle the time lapse indicator 111. In such an embodiment, the actuator performs as a skirt relative to the time lapse indicator 111 and by making the actuator transparent or otherwise by including one or more see-through sections, the state of the time lapse indicator 111 may be inspected from the exterior of the device. The time lapse indicator 111 and the actuator 108 may be so dimensioned that the actuator 108 exerts a shear force on the pressure sensitive substance of the time lapse indicator 111 upon actuation of the actuator, to thereby register operation of the actuator.

In addition to directly identifying a dose administration, i.e. the delivering or expelling action, various other actions which associates with the action of administering a dose may be used as an indication of a recently performed dose administration. For instance, as a non-exhaustive list of actions, and in accordance with the particular type of medical delivery device, actions such as the procedure of operating a dosage selector, mounting or removal of a cap, the procedure of “arming” of an injector prior to “firing” the injector, the mounting or removal of a needle from the device, the procedure of inserting an injection needle into the skin of the user or, alternatively, the procedure of removing the needle from the skin may be used as to identify the administration of a dose.

A third embodiment of a medical delivery device is schematically shown in FIG. 2c, where an injection pen 100 again incorporates a cap 110 for protecting a needle cannula 103 and a cartridge accommodated in the injection pen 100. In the shown state of injection pen 100, the cap 110 is shown in a detached or non-protecting state. Since the removal of cap 110 from the remainder of the injection device 100 is a prerequisite for carrying out the administration process, the identification that the cap 110 has been detached from main housing 104 or that it has been re-attached can be used as an indication as to whether or not an administration has been carried out within the previous pre-defined time-frame. Hence, a time lapse indicator of the above described kind may be associated with the cap 110, such as the interface between cap 110 and the main housing 104. In the depicted form, the time lapse indicator 111 is attached to or formed into cap 110. The piezochromic substance of time lapse indicator 111 is exerted to pressure either by the user applying a directly acting force to the substance by gripping around the cap 110 with a hand, or alternatively, as the cap 110 wall material may be designed to deform when the cap detaches or attaches to the main housing, e.g. by means of a snap fitting, the resulting pressure is applied indirectly to the piezochromic substance. Hence, after administration of a dose, where the cap has been removed and re-attached, the time lapse indicator has changed to a colour different than the initial colour for a pre-determined time-interval. Hence, the occurrence of a dose administration can be checked retrospectively.

In a still further embodiment (not shown), a window in housing 104, which correspond to window 109 depicted in FIG. 2c, may provide views to an associated time lapse indicator of the same kind as referred to in the above embodiments. The time lapse indicator may be associated with a dose dial scale which is viewable through the window such that the window in addition to conveying information as to selected dosage amounts provide information as to whether or not an injection has been performed within the lapse of a time interval corresponding to the pre-defined time interval for the time lapse indicator. For example, the time lapse indicator may be associated with the “0”-marking on the dose dial scale, either superposed on or positioned next to the “0”-marking, such that the user may immediately check in window whether or not an injection has recently been performed.

In FIG. 3a a needle assembly 200 according to a fourth embodiment is schematically shown, the needle assembly being intended for cooperation with an associated medical delivery device, such as an injection pen, during an administering action. The needle assembly 200 includes a needle hub section 212 which is adapted to releasably connect to the associated injection device. Fixedly connected with needle hub section 212 is a needle cannula 203 which includes a pointed tip at its distal end. Slideably arranged with respect to needle hub section 212 is a needle shield 213. Needle shield 213 is in the depicted embodiment transparent. In FIG. 3a, needle shield 213 is positioned in a distal position which defines a shielded state of the needle assembly. In this state the needle shield 213 covers needle cannula 203, in particular to avoid accidental needle sticks. By sliding needle shield 213 relatively to needle hub section 212, the needle shield may be brought into a proximal position where the needle cannula front end is exposed. This state defines an exposed state of the needle assembly 200. In the depicted embodiment, the needle assembly 200 may further comprise a biasing mechanism (not shown) for biasing the needle shield 213 towards the distal direction. In other embodiments, the needle assembly 200 is provided with a locking mechanism (not shown) for locking the needle shield in the distal direction subsequent to performing an administration.

When an injection is to be performed, the injection device including needle assembly 200 is pressed towards the skin of the user, which causes the needle shield 213 to slide proximally while leaving the needle cannula 203 free to pierce the skin of the user. After administration, the needle cannula 203 is retracted from the skin, causing the needle shield 213 to slide back to its distal position, leaving the needle cannula 203 surrounded by needle shield 213.

Needle hub section 212 is provided with a time lapse indicator 211 which works in a way corresponding to the embodiment shown in FIG. 2c. In the FIG. 3a embodiment, the time lapse indicator 211 including a piezochromic substance is attached to or formed into a wall section of the needle hub section 212. When needle shield 213 moves proximally or distally, the piezochromic substance is exerted to pressure due to being manipulated by needle shield 213 causing the time lapse indicator to change to a colour different than the initial colour for a pre-determined time-interval.

FIG. 3b shows a fifth embodiment which relates to the fourth embodiment shown in FIG. 3a. Again, a needle assembly 200 is shown, which mechanically correspond to the fourth embodiment. Instead of time lapse indicator 211 being associated with the wall section of needle hub section 212, time lapse indicator is associated with needle shield 213, e.g. attached to or formed into a wall section of needle shield 213. In this embodiment, the piezochromic substance of time lapse indicator 211 is exerted to mechanical stress or pressure when the needle shield 213 moves relatively to needle hub section 212, thereby causing the time lapse indicator to change to a colour different than the initial colour for a pre-determined time-interval.

In still other embodiments of the invention (not shown), the time lapse indicator may be associated with a container for a needle, where the container includes a time lapse indicator for indicating recent removal and/or insertion of a needle from/into the container. The container may alternatively be formed as a needle magazine having a plurality of compartments for holding a plurality of injection needles and where a time lapse indicator may indicate whether or not a needle has been removed or inserted within the preceding time interval. A single time lapse indicator may be associated with a common interface for coupling the plurality of injection needles in turn to an associated injection device, or alternatively, a time lapse indicator may be provided for each particular needle compartment of the needle magazine to indicate recent usage of each particular needle.

In all the above embodiments, the administering indication returns to its initial or an alternative state (colour or opacity) after a pre-determined period of time (ranging from a few minutes to hours), thus retrospectively indicating use of the medical delivery device for administering a dose of a drug. Within the context of the present invention, additional or alternative optical property changes can be incorporated in the time lapse indicator without departing from the scope of the invention. In embodiments where the delivery device or the needle assembly is to be used for several distinct administrations, the above is repeated every time a dose is delivered, e.g. when the injection button is pushed, the cap is removed or re-attached or the needle is used. It is recognized that the shown embodiments may incorporate a time lapse indicator wherein the third visual state of the indicator is dissimilar from the first visual state, e.g. has a different colour in the third state than in the first state. Such indicators may for example be used in devices intended for single use only.

FIGS. 7a, 7b and 7c shows an exemplary embodiment blister package 300 according to the fifth aspect of the present invention. The blister package includes a first material portion which forms a first layer defining a plurality of cavities and a lid layer, the two layers being bonded to each other at peripheral portions of each cavity to thereby form a plurality of separate compartments. Each compartment sealingly accommodates a material portion, such as a medicament pill or tablet 301. When a user presses onto one of the compartments, the pill or tablet contained therein is dispensed from the blister package (as seen in FIG. 7a).

A time lapse indicator, which in the depicted embodiment comprises a reversible piezochromic material, is associated with each respective compartment so that upon rupturing the lid portion in question, the time lapse indicator associated with that particular compartment changes from a first state to a second state. This state change may be associated with a change in a visual property such as colour or opacity. Due to the controlled reversal time of the piezochromic material, the piezochromic substance remains in the second visual state during a pre-defined time-interval subsequent to the action of dispensing the pill or tablet from the compartment. Upon expiry of the pre-defined time-interval, the piezochromic material returns to the first visual state or alternatively changes to a third visual state which is immediately discernible from the second visual state.

As seen FIG. 7b, the first material portion forming the cavities includes a piezo-chromic material. It follows from the colour markings of the compartments 302, 303 and 304 that the compartment 304 has been accessed within the preceding pre-defined time interval, whereas the compartments 302 and 303 have been accessed prior to said pre-defined time interval. It is readily acknowledged that the remaining compartments not yet have been accessed. The colour state of compartment 304 indicates that an administration has been performed within the preceding pre-defined time interval and that further administrations should be postponed at least until the colour indicator associated with compartment 304 returns to its initial state. In this way a potentially harmful double medication can be prevented.

FIG. 7c shows a further embodiment of a blister package. However in this embodiment the time lapse indicators comprising the piezo-chromic material are associated with the lid layer that is adapted to rupture upon dispensing pills or tablets from the blister package.

The piezo-chromic material may be selected so that the reversal time of the time lapse indicators matches the administration scheme of the particular medication in question. Non-limiting exemplary reversal times may be selected such as 10 minutes, 30 minutes, 1 hour, 3 hours, 6 hours, 12 hours or 24 hours. Other reversal times may also be chosen.

In accordance with a further aspect of the invention, different time delay or time lapse indicators having more than two state changes will next be described. These time lapse indicators may be used in addition or to replace the time lapse indicators described above. FIG. 4 shows such time lapse indicator having a plurality of closely intermeshing dots of respective first and second types of piezo-chromic material. By initially rubbing the surface of the time lapse indicator, both types of piezo-chromic materials are activated to visually change its appearance to a respective second state. After expiry of a first pre-defined time interval, the first piezo-chromic material returns to its initial state, whereas the second piezo-chromic material returns to its initial state somewhat later after expiration of a second time interval. By appropriately choosing the visual state changes of each of the piezo-chromic materials, the time indicator may visually indicate three different states, e.g. a first colour prior to activation, a second colour when the time lapse indicator is activated and a third colour when the first time interval has expired. The time lapse indicator returns to the initial colour after expiration of the second time interval has expired. A schematic representation of this effect is shown in FIG. 5a. Further piezo-chromic materials may be incorporated by intermeshing with the other piezo-chromic materials to thereby obtain further state changes.

A second embodiment in accordance with the sixth aspect of the present invention incorporates a plurality of time lapse indicators arranged as a plurality of segments. FIG. 5b shows such indicators having four individual segments arranged as annular segments of a circle. In the shown embodiment, each individual segment comprises a piezo-chromic material having a distinct state reversal time as compared to the others. The different time delay intervals may be chosen in accordance with the particular application in question. By appropriately choosing the piezo-chromic materials, the approximate time elapsed between each segment state change may be chosen as 30 minutes intervals, hour intervals, 2-hour intervals, 3-hour intervals, 4-hour intervals, 6-hour intervals or twelve hour intervals, said time intervals providing non-limiting examples of the invention. In these examples, the particular group of segments provide a linear scale of the time elapsed since activation. However, also non-linear scales may be provided having non-uniform expiry steps. Alternatively to the circular/annular representation shown in FIG. 5b, a concentric type representation may be used, e.g. a central segment and one or more additional ring shaped segments arranged concentrically with respect to the central segment. Also linear scales may be chosen or any other shape.

Also, the principle set forth in FIG. 4 and FIG. 5a may be employed in one or more segments of a segmentized time delay indicator. It should be noted that the two embodiments of FIGS. 5a and 5b only shows the possible different states of the respective scale-type time delay indicators. Embodiments may include scale-type time delay indicators which do not return to its initial state as well as scale-type time delay indicators which do return to its initial state. The latter type mentioned may be of the reactivatable kind which enables repetitive use.

FIG. 6 show an application where two different messages appear superposed, i.e. overlapping in space, such that the first message is shown prior to activation of the time delay indicator, and the second message appear directly after activation almost simultaneously as the first message disappears. Alternatively, the said change happens after expiry of a pre-defined time interval. Still alternatively, the first message disappears at activation of the time delay indicator whereas the second message appears after lapse of the pre-defined time interval. These message-type indicators may be provided by having intermeshed portions of different piezo-chromic materials as explained above in connection with FIG. 4.

The time lapse indicators of the sixth aspect may be used in connection with any other embodiment of the other aspects of the invention.

Some preferred embodiments have been shown in the foregoing, but it should be stressed that the invention is not limited to these, but may be embodied in other ways within the subject matter defined in the following claims. The figures e.g. discloses medical delivery systems of the present invention in the form of an injection pen, however, this particular delivery device and its shape is in no way limiting for the present invention as defined in the claims.

1. A handheld medical delivery device for accommodating a drug and for administering one or more doses of the drug to a subject user, the medical delivery device comprising:
an actuator for delivering a dose of the drug from an outlet of the medical delivery device, and
a time lapse indicator adapted to be in a first state prior to an action associated with administering a dose, to change to a second state in response to said action and to change to a third state after lapse of a pre-defined time interval subsequent to said action, said pre-defined time interval being longer than 1 minute, the time lapse indicator providing a visually perceivable indication of the state of the indicator, and
wherein the time lapse indicator comprises a pressure sensitive substance exhibiting a change in a visual property when exerted to pressure and/or force and wherein the medical delivery device is configured to cause the pressure sensitive substance to change from said first state to said second state responsive to a user performing said action.

2. A medical delivery device as defined in claim 1, wherein, when changing from said first to said second state, the pressure sensitive substance is adapted to alter one or more properties selected from the group consisting of colour, absorptivity, reflectivity, transmissivity, diffusivity and polarization when being exerted to a change in pressure and/or force.

3. A medical delivery device as defined in claim 1, wherein the pressure sensitive substance comprises a piezochromic material.

4. A medical delivery device as defined in claim 1, wherein the state change of said indicator is reversible so that said third state correspond to said first state.

5. A medical delivery device as defined in claim 1, wherein the state change of said indicator is reactivatable so that said pressure sensitive substance is changeable from the first state to the second state by a user repeatedly performing said action.

6. A medical delivery device as defined in claim 1, wherein the indicating member is configured to substantially maintain the visual property of the second state during lapse of the pre-defined time interval.

7. A medical delivery device as defined in claim 1, wherein the pre-defined time interval is longer than 5 minutes, more preferably longer than 15 minutes, more preferably longer than 30 minutes, more preferably longer than 1 hour, more preferably longer than 2 hours, more preferably longer than 5 hours, more preferably longer than 10 hours, more preferably longer than 20 hours and most preferably longer than 36 hours.

8. A medical delivery device as defined in claim 1, wherein said action is defined as the actuation of the actuator and wherein the pressure sensitive substance is configured so that actuation of the actuator causes the pressure sensitive substance to change from the first state to the second state.

9. A medical delivery device as defined in claim 8, wherein the actuator is adapted to move in a distal direction when actuated, and where the pressure sensitive substance is arranged on a proximally facing part of the actuator.

10. A medical delivery device as defined in claim 1, wherein the medical delivery device further comprises a dose size selector which is operable to set the size of a dose and that said action is defined as the operation of the dose size selector and wherein the pressure sensitive substance is configured so that operation of the dose size selector causes the pressure sensitive substance to change from the first state to the second state.

11. A medical delivery device as defined in claim 1, wherein the medical delivery device further comprises a cap which can be moved from a protecting state where the outlet is protected during storage of the device to a non-protected state where the outlet is exposed for administering a dose of the drug and wherein said action is defined as the moval of the cap from the protecting state to the non-protecting state and/or from the non-protecting state to the protecting state.

12. A medical delivery device as claim 1, wherein the outlet comprises a needle mount for connecting to an injection needle and optionally comprises an injection needle connected to said needle mount, wherein the medical delivery device further comprises a needle shield and wherein the injection needle, when mounted, and the needle shield is configured to move relative to each other when the injection needle is inserted into and/or removed from the skin of the subject user and wherein said action is defined as the said insertion and/or removal of the injection needle into/from the skin of the subject user.

13. An injection needle assembly for cooperation with a medical delivery device, wherein the injection needle assembly comprises:
a needle cannula having a distal end for penetrating the skin of a subject user and a needle shield, wherein the needle assembly is positionable in a shielded state where the distal end of the needle cannula is shielded by the needle shield and in an exposed state where the distal end of the needle cannula is exposed,
a visual indicator adapted to change from a first state to a second state responsive to the needle assembly changing from said shielded state to the exposed state or vice versa,
wherein the visual indicator is configured for indicating time elapsed since said change from the first state to the second state, wherein the visual indicator incorporates a pressure sensitive substance exhibiting a change in a visual property when exerted to pressure or force, and wherein the visual indicator changes said visual property subject to the needle assembly changing from the shielded state to the exposed state or vice versa, said pressure sensitive substance exhibiting a further change in a visual property after lapse of a pre-defined time interval subsequent to said change from the first state to the second state.

14. Use of a pressure sensitive substance in a medical delivery device for signalling time elapsed since administering a drug from the medical delivery device, wherein the pressure sensitive substance exhibits a change in a visual property when exerted to pressure or force, wherein the pressure sensitive substance is adapted to change from a first state to a second state responsive to a user performing an action associated with administering a dose, and wherein the pressure sensitive substance exhibits a further change in a visual property after lapse of a pre-defined time interval subsequent to said change from the first state to the second state.

15. A method of providing a medical delivery device, the method comprising the steps of:
providing a medical delivery device, the device comprising an actuator for actuating the administering of a dose of a drug from the medical delivery device, and
applying a time lapse indicator comprising a pressure sensitive substance exhibiting a reversible change in a visual property when exerted to pressure or force and exhibiting a further change in a visual property upon lapse of a pre-determined time interval, the pressure sensitive substance being configured to be activated to change the visual property by an action associated with the administering of a dose.
